(19) United States
(12) Patent Application Publication
(10) Pub. No.: US 2013/0144167 A1
(43) Pub. Date: Jun. 6, 2013

(54) LESION DIAGNOSIS APPARATUS AND METHOD USING LESION PERIPHERAL ZONE INFORMATION

(71) Applicants: Jae-Cheol LEE, Seoul (KR); Kyoung-Gu Woo, Seoul (KR); Moon-Ho Park, Seoul (KR)

(72) Inventors: Jae-Cheol LEE, Seoul (KR); Kyoung-Gu Woo, Seoul (KR); Moon-Ho Park, Seoul (KR)

(21) Appl. No.: 13/527,712

(22) Filed: Jun. 20, 2012

(30) Foreign Application Priority Data
Dec. 2, 2011 (KR) 10-2011-0128594

(51) Int. Cl. A61B 8/13

(52) U.S. Cl. 600/443

(57) ABSTRACT

A lesion diagnosis apparatus is provided. The lesion diagnosis apparatus using lesion peripheral zone information includes a region division unit configured to divide a region based on lesions or organs included in a medical image, a region exclusion unit configured to exclude regions including lesions or organs other than a diagnostic target lesion among regions divided by the region division unit, and a peripheral region determination unit configured to determine regions other than a diagnostic region among remaining regions other than regions excluded by the region exclusion unit, as peripheral regions for lesion diagnosis.

### CROSS REFERENCE TO RELATED APPLICATION

This application claims the benefit under 35 U.S.C. §119(a) of a Korean Patent Application No. 10-2011-0128594, filed on Dec. 2, 2011, in the Korean Intellectual Property Office, the entire disclosure of which is incorporated herein by reference for all purposes.

### BACKGROUND

1. Field

The following description relates to a lesion diagnosis apparatus and a method using lesion peripheral zone information.

2. Description of the Related Art

Lesions can be detected or diagnosed using ultrasonic wave images. In response to ultrasonic waves being completely reflected or absorbed inside bodily tissues, an image of a rear tissue of the body does not appear.

However, as a difference between impedance of the two tissues increases, an amount of reflected waves increase and an amount of penetrated waves decrease. Accordingly, an acoustic shadow is generated in rear portions of two boundary surfaces. The generation of the acoustic shadow is referred to as a posterior acoustic shadow (PAS), and the PAS can be seen at bones in the body, visceral gases, gallstones and the like.

Various types of lesions may be diagnosed using such a phenomenon. In this case, if there is a cystic mass in the body, ultrasonic waves directly pass through a cystic portion without weakening, and are reflected and attenuated at a solid tissue of the cystic portion to then increase echoes in a rear portion of the cystic mass. This increase in echoes is referred to as a posterior acoustic enhancement.

However, in response to another lesion existing at the bottom of the lesion, or multiple lesions overlapping, difficulty in diagnosing the lesion using posterior acoustic enhancement may exist. Accordingly, technology that can more accurately diagnose the lesion using peripheral zone information is being studied.

### SUMMARY

In one general aspect, a lesion diagnosis apparatus is provided. The lesion diagnosis apparatus using lesion peripheral zone information includes a region division unit configured to divide a region based on lesions or organs included in a medical image, a region exclusion unit configured to exclude regions including lesions or organs other than a diagnostic target lesion among regions divided by the region division unit, and a peripheral region determination unit configured to determine regions other than a diagnostic region among remaining regions other than regions excluded by the region exclusion unit, as peripheral regions for lesion diagnosis.

The region division unit may divide the region into units of imaginary quadrangles in which the lesions and organs are inscribed.

The region exclusion unit may further exclude rear regions of the regions including lesions or organs other than the diagnostic target lesion.

The peripheral region determination unit may select regions located on the rear of the diagnosis region and on the left and right of the rear of the diagnosis region, as peripheral regions, among regions further excluding the rear regions of the regions including lesions or organs other than the diagnostic target lesion.

The lesion diagnosis apparatus may further include an analysis unit configured to analyze medical image signals of the peripheral regions determined by the region determination unit, and a diagnosis unit configured to diagnose a lesion included in the diagnostic region based on a comparison of medical image signals of the peripheral regions analyzed by the analysis unit.

The analysis unit may calculate an average value and a standard deviation of the medical image signals of the peripheral regions and correct the peripheral regions by excluding peripheral regions in which the standard deviation exceeds a reference value.

The analysis unit may analyze change amounts of the medical image signals of the peripheral regions located on the rear of the diagnostic region and on the left and right of the rear of the diagnostic region on the basis of a center of the diagnostic region.

The change amount of the medical image signals may be a difference in intensity between the medical images.

The change amount of the medical image signals may be a difference in brightness between the medical images.

The analysis unit may include a rear region analysis unit configured to analyze medical image signals of the peripheral regions located on the rear of the diagnostic region, a left and right region analysis unit configured to analyze medical image signals of the peripheral regions located on the left and right of the rear of the diagnostic region, and a peripheral region correction unit configured to calculate an average value and a standard deviation of the medical image signals of the peripheral regions analyzed by the rear region analysis unit or the left and right region analysis unit and correct peripheral regions by excluding peripheral regions in which the standard deviation exceeds a reference value.

The diagnosis unit may diagnose the lesion included in the diagnostic region based on a comparison of medical image signals of the peripheral regions located on the rear of the diagnostic region and on the left and right of the rear of the diagnostic region.

The diagnosis unit may determine whether the lesion included in the diagnostic region is a cystic mass or a solid mass based on a comparison of change amounts of the medical image signals of the peripheral regions located on the rear of the diagnostic region and on the left and right of the rear of the diagnostic region.

The diagnosis unit may determine whether the lesion included in the diagnostic region is a cystic mass or a solid mass based on a comparison of differences in intensity between the medical image signals of the peripheral regions located on the rear of the diagnostic region and on the left and right of the rear of the diagnostic region.

The diagnosis unit may determine the lesion included in the diagnostic region as a cystic mass in response to intensity of the medical images of the peripheral regions located on the rear of the diagnostic region being greater than intensity of the medical images of the peripheral regions located on the left and right of the rear of the diagnostic region.

The diagnosis unit may determine the lesion included in the diagnostic region as a solid mass in response to intensity of the medical images of the peripheral regions located on the rear of the diagnostic region being less than intensity of the medical images of the peripheral regions located on the left and right of the rear of the diagnostic region.

The diagnosis unit may determine whether the lesion included in the diagnostic region is a cystic mass or a solid mass based on a comparison of differences in brightness between the medical image signals of the peripheral regions located on the rear of the diagnostic region and on the left and right of the rear of the diagnostic region.

The diagnosis unit may determine the lesion included in the diagnostic region as a cystic mass in response to the brightness of the medical images of the peripheral regions located on the rear of the diagnostic region being higher than that of the medical images of the peripheral regions located on the left and right of the rear of the diagnostic region.

The diagnosis unit may determine the lesion included in the diagnostic region as a solid mass in response to the brightness of the medical images of the peripheral regions located on the rear of the diagnostic region being lower than that of the medical images of the peripheral regions located on the left and right of the rear of the diagnostic region.

The diagnosis unit may diagnose the lesion included in the diagnostic region based on a comparison of an average value of medical image signals of the peripheral regions located on the rear of the diagnostic region and an average value of medical image signals of the peripheral regions located on the left and right of peripheral regions located on the rear of the diagnostic region.

A lesion or organ of the region may be surrounded by sides of a quadrangle.

Sides of a quadrangle may surround the diagnostic target lesion based on horizontal and vertical lengths of the diagnostic target lesion.

In another aspect, a lesion diagnosis method is provided. The lesion diagnosis method using lesion peripheral zone information includes dividing a region based on lesions or organs included in a medical image, excluding regions including lesions or organs other than a diagnostic target lesion among divided regions, and determining regions other than a diagnosis region including the diagnostic target lesion among remaining regions after excluding the regions including lesions or organs other than the diagnostic target lesion among the divided regions, as peripheral regions for lesion diagnosis.

The lesion diagnosis method may further include analyzing medical image signals of the determined peripheral regions, and diagnosing a lesion included in the diagnostic region based on a comparison of medical image signals of the analyzed peripheral regions.

The lesion diagnosis method may further include calculating an average value and a standard deviation of the medical image signals of the determined peripheral regions and correcting peripheral regions by excluding peripheral regions in which the standard deviation exceeds a reference value.

Other features and aspects may be apparent from the following detailed description, the drawings, and the claims.

### BRIEF DESCRIPTION OF THE DRAWINGS

FIG. 1 is an overview of an example of a computer aided diagnosis (CAD) system for lesion diagnosis.

FIG. 2 is a diagram illustrating an example of a lesion diagnosis apparatus using lesion peripheral zone information.

FIG. 3 is a view illustrating an example of a region divided by a lesion diagnosis apparatus using lesion peripheral zone information.

FIG. 4 is a flowchart illustrating an example of a lesion diagnosis method using lesion peripheral zone information.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be suggested to those of ordinary skill in the art. Also, descriptions of well-known functions and constructions may be omitted for increased clarity and conciseness.

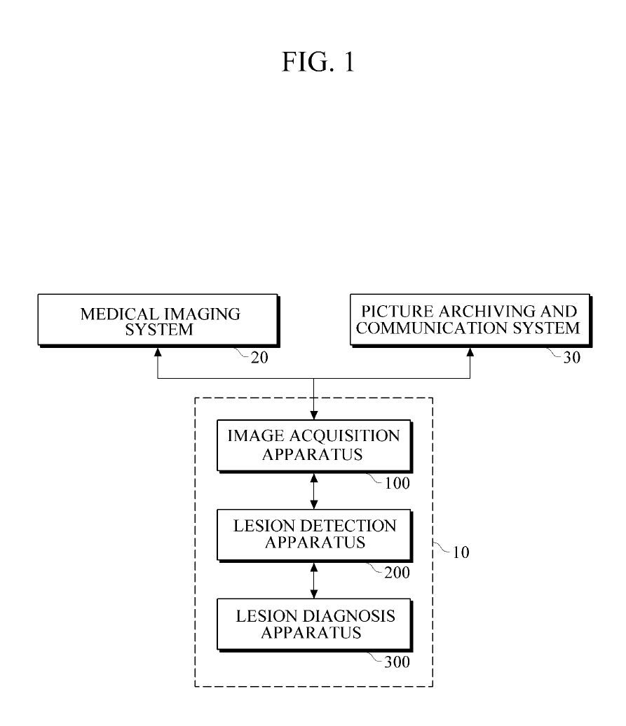

FIG. 1 illustrates an example of a computer aided diagnosis (CAD) system for lesion diagnosis. The CAD system 10 includes an image acquisition apparatus 100, a lesion detection apparatus 200, and a lesion diagnosis apparatus 300. The image acquisition apparatus 100, the lesion detection apparatus 200 and the lesion diagnosis apparatus 300 may be a single logical or physical device or independent logical or physical devices.

The image acquisition apparatus 100 may use a medical imaging system (MIS) 20 or a picture archiving and communication system (PACS) 30, etc to obtain medical images. The medical images may be, for example, ultrasonic images of a body.

The lesion detection apparatus 200 may detect lesions or organs in the body from a medical image obtained by the image acquisition apparatus 100. For example, the lesion detection apparatus 200 may use a region of interest (ROI) method to detect lesions or organs in the body from the medical image.

The lesion diagnosis apparatus 300 may diagnose a lesion detected by the lesion detection apparatus 200. For example, the lesion diagnosis apparatus 300 may determine whether the lesion is a cystic mass or solid mass, and also may determine whether a tumor is malignant or benign.

The lesion diagnosis apparatus 300 of the CAD system 10 may improve an accuracy of the diagnosis of the lesion. The improved lesion diagnosis apparatus 300 may diagnose a lesion using peripheral zone information of the lesion. In addition, the improved lesion diagnosis apparatus 300 may diagnose a lesion that is a target of diagnosis where other lesions or organs around the target lesion that may affect the diagnosis are excluded. Thus, the target lesion may be more accurately diagnosed.

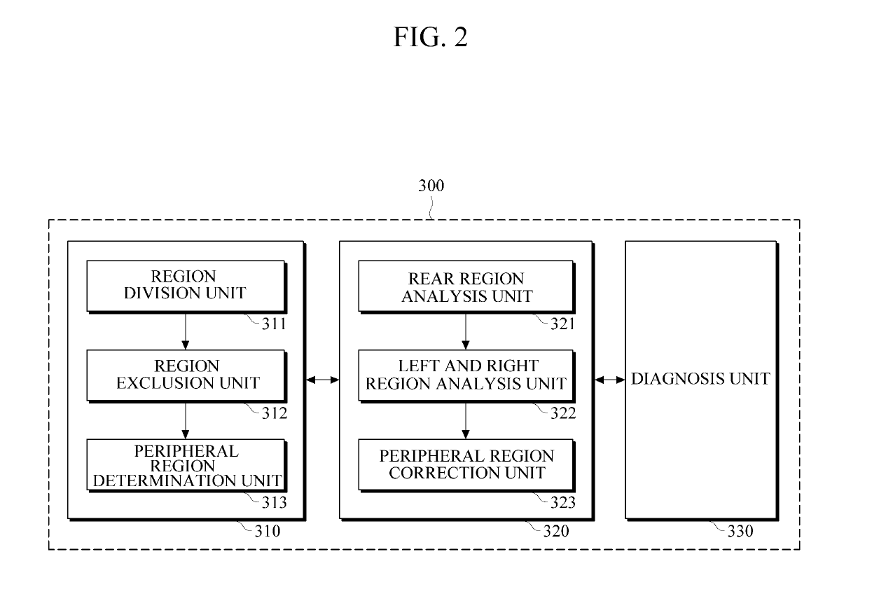

FIG. 2 illustrates an example of a lesion diagnosis apparatus using lesion peripheral zone information.

The example of the lesion diagnosis apparatus 300 using lesion peripheral zone information includes a region determination unit 310, an analysis unit 320 and a diagnosis unit 330.

The region determination unit 310 may divide a region based on lesions or organs included in a medical image. The region determination unit 310 may exclude regions including lesions or organs other than the diagnostic target lesion among the divided regions. The region determination unit 310 may determine remaining, as peripheral regions for lesion diagnosis, regions other than a diagnosis region including the diagnostic target lesion.

The region determination unit 310 includes a region division unit 311, a region exclusion unit 312 and a peripheral region determination unit 313. The region division unit 311 may divide a region based on lesions or organs included in a medical image.

The region exclusion unit 312 may exclude regions including lesions or organs other than the diagnostic target lesion among the divided regions.

The peripheral region determination unit 313 may determine, as peripheral regions for lesion diagnosis, regions other than the diagnosis region among the remaining regions other than the regions excluded by the region exclusion unit 312. The organs may be determined using anatomic information.

For example, the region division unit 311 may divide a region into imaginary quadrangles, each quadrangle including a lesion or organ inscribed therein. In response to the lesions and organs being included in the medical image, the lesion detection apparatus 200 may calculate horizontal and vertical lengths of the lesions or organs. Accordingly, the region determination unit 310 may form an imaginary quadrangle on the sides of the lesion or organ. The sides of the imaginary quadrangle may correspond to the horizontal and vertical lengths of the lesion or organ and include the lesion or organ inscribed therein. Thus, the region determination unit 310 may divide a region into such imaginary quadrangles.

Meanwhile, the region exclusion unit 312 may exclude rear regions of regions including the lesions or organs other than the diagnostic target lesions. Since a posterior acoustic shadow feature due to the lesions may affect the excluded rear regions of regions including the lesions or organs other than the diagnostic target lesions, the excluded rear regions may influence diagnosis of the diagnostic target lesions. Accordingly, in order to accurately diagnose lesions, the rear regions of regions including the lesions or organs other than the diagnostic target lesions may be excluded.

Meanwhile, the peripheral region determination unit 313 may select, as peripheral regions, regions located on the rear of the diagnosis region and on the left and right of the rear of the diagnosis region. The peripheral regions may be among regions excluding the rear regions of the regions including lesions or organs other than the diagnostic target lesion. In other words, since regions located on the front of the diagnostic region and on the left and right of the front of is the diagnostic region may not be related to having the posterior acoustic shadow feature, the region determination unit 310 may select, as the peripheral regions, regions located on the rear of the diagnostic region and on the left and right of the rear of the diagnostic region.

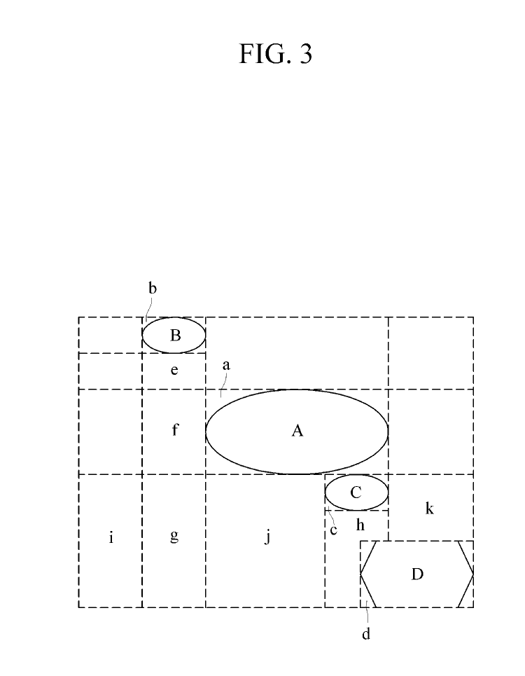

FIG. 3 illustrates an example of a medical image divided by a lesion diagnosis apparatus using lesion peripheral zone information. In the example of FIG. 3, ‘A’ represents a diagnostic target lesion, ‘B’ and ‘C’ represent lesions on the periphery of the diagnostic target lesion, and ‘D’ represents an organ.

Referring to FIG. 3, a medical region is divided into units of quadrangles in which a diagnostic target lesion ‘A,’ lesions ‘B’ and ‘C’ that are on the periphery of the diagnostic target lesion, and an organ ‘D’ are inscribed.

Meanwhile, a region ‘a’ represents a diagnostic region including a diagnostic target lesion, regions ‘b’ and ‘c’ represent regions each including lesions that are on the periphery of the diagnostic target lesion, a region ‘d’ represents a region including an organ, and regions ‘e,’ ‘f,’ ‘g,’ and ‘h’ represent rear regions of regions. Each of the rear regions of regions include lesions on the periphery of the diagnostic target lesion.

In FIG. 3, regions ‘b,’ ‘c,’ ‘d,’ ‘e,’ ‘f,’ ‘g,’ and ‘h’ may affect the diagnosis of the diagnostic target lesion, and regions ‘i,’ ‘j,’ and ‘k’ are located on the rear of the diagnostic regions and on right and left of the rear of the diagnostic regions. Regions ‘b,’ ‘c,’ ‘d,’ ‘e,’ ‘f,’ ‘g,’ and ‘h’ may be excluded by the region determination unit 310. Regions ‘i,’ ‘j,’ and ‘k’ may be selected as the peripheral regions.

The analysis unit 320 may analyze medical image signals of the peripheral regions. The peripheral regions may be determined by the region determination unit 310. In this case, the analysis unit 320 may calculate an average value and a standard deviation of medical image signals of the peripheral regions, respectively, and the analysis unit 320 may exclude peripheral regions in response to the standard deviation exceeding a reference value to correct peripheral regions.

The analysis unit 320 includes a rear region analysis unit 321, a left and right region analysis unit 322, and a peripheral region correction unit 323. The rear region analysis unit 321 may analyze medical image signals of the peripheral regions located on the rear of the diagnostic region. The left and right region analysis unit 322 may analyze medical image signals of the peripheral regions located on the left and right of the diagnostic region.

The peripheral region correction unit 323 may calculate an average value and a standard deviation of medical image signals of the peripheral regions and correct peripheral regions. The peripheral regions may be corrected by excluding peripheral regions where the standard deviation exceeds a reference value.

By correcting the peripheral regions, an increase in an accuracy of lesion diagnosis may occur. In other words, in response to a great difference between medical image signals of a particular peripheral region and other peripheral regions due to noise signals, an increase in the accuracy of lesion diagnosis may occur by excluding regions having the great difference.

For example, the analysis unit 320 may analyze amounts of change of the medical image signals of the peripheral regions located on the rear of a diagnostic region and on the left and right of the rear of the diagnostic region on the basis of a center of the diagnostic region. In this case, the amounts of change of the medical image signals may correspond to differences in intensity or brightness between the medical images

The diagnosis unit 330 may compare medical image signals of peripheral regions analyzed by the analysis unit 320 to diagnose a lesion in the diagnostic region. For example, the diagnosis unit 330 may diagnose a lesion included in a diagnostic region based on a comparison of medical image signals of the peripheral regions located on the rear of the diagnostic region and image signals of the peripheral regions located on the left and right of the is rear of the diagnostic region.

In this case, the diagnosis unit 330 may diagnose a lesion in a diagnostic region based on a comparison of an average value of medical image signals of the peripheral regions located on the rear of the diagnostic region and an average value of image signals of the peripheral regions located on the left and right of the rear of the diagnostic region.

Referring to FIG. 3, the diagnosis unit 330 compares medical signals in a peripheral region ‘j’ and peripheral regions ‘i’ and ‘k’. The peripheral region ‘j’ is located on the rear of the diagnostic region. The peripheral regions ‘i’ and ‘k’ are located on the left and right of the rear of the diagnostic region.

A difference between amounts of change of medical image signals of peripheral regions located on the rear of the diagnostic region and on the left and right of the rear of the diagnostic region may occur due to a diagnostic target lesion included in the diagnostic region.

Accordingly, the diagnosis unit 330 may compare amounts of change of medical image signals of peripheral regions located on the rear of the diagnostic region and on the left and right of the rear of the diagnostic region to determine whether a lesion included in the diagnostic region is a cystic mass or a solid mass.

For example, the diagnosis unit 330 may compare differences in intensity between medical images of peripheral regions located on the rear of the diagnostic region and on the left and right of the rear of the diagnostic region to determine whether a lesion included in the diagnostic region is a cystic mass or a solid mass.

In this case, in response to an intensity of medical images of peripheral regions located on the rear of the diagnostic region being greater than an intensity of the medical images of the peripheral regions located on the left and right of the rear of the diagnostic region, the diagnosis unit 330 may determine a lesion included in the diagnostic region as a cystic mass.

Meanwhile, in response to an intensity of the medical images of peripheral regions located on the rear of the diagnostic region being less than an intensity of the medical images of the peripheral regions located on the left and right of the rear of the diagnostic region, the diagnosis unit 330 may determine the lesion included in the diagnostic region as a solid mass.

For example, the diagnosis unit 330 may determine whether a lesion included in the diagnostic region is a cystic mass or a solid mass based on a comparison of differences in brightness between medical images of peripheral regions located on the rear of the diagnostic region and on the left and right of the rear of the diagnostic region.

In this case, in response to a brightness of the medical images of peripheral regions located on the rear of the diagnostic region being higher than a brightness of the medical images of the peripheral regions located on the left and right of the rear of the diagnostic region, the diagnosis unit 330 can determine a lesion included in the diagnostic region to be a cystic mass.

Meanwhile, in response to the brightness of the medical images of peripheral regions located on the rear of the diagnostic region being lower than the brightness of the medical images of peripheral regions located on the left and right of the rear of the diagnostic region, the diagnosis unit 330 may determine a lesion included in the diagnostic region to be a solid mass.

Accordingly, in response to the lesions in the medical image being diagnosed using a posterior acoustic shadow feature (PASF) method, an accuracy of the lesion diagnosis in a state where overlapping lesions are present or even acoustic interference is present may be increased due to peripheral lesions.

In addition, a consideration of a posterior acoustic effects of lesions located on the bottom thereof as well as the periphery thereof may be used to diagnose a lesion. In response to an organ being on a rear of a lesion or on a periphery of the rear of the lesion, a lesion may be diagnosed without influence of the organ.

Therefore, a diagnostic target lesion may be diagnosed in a state in which lesions or organs around a diagnostic target lesion affecting lesion diagnosis are excluded. Accordingly, the lesion may be accurately diagnosed and thus diagnostic reliability may be improved.

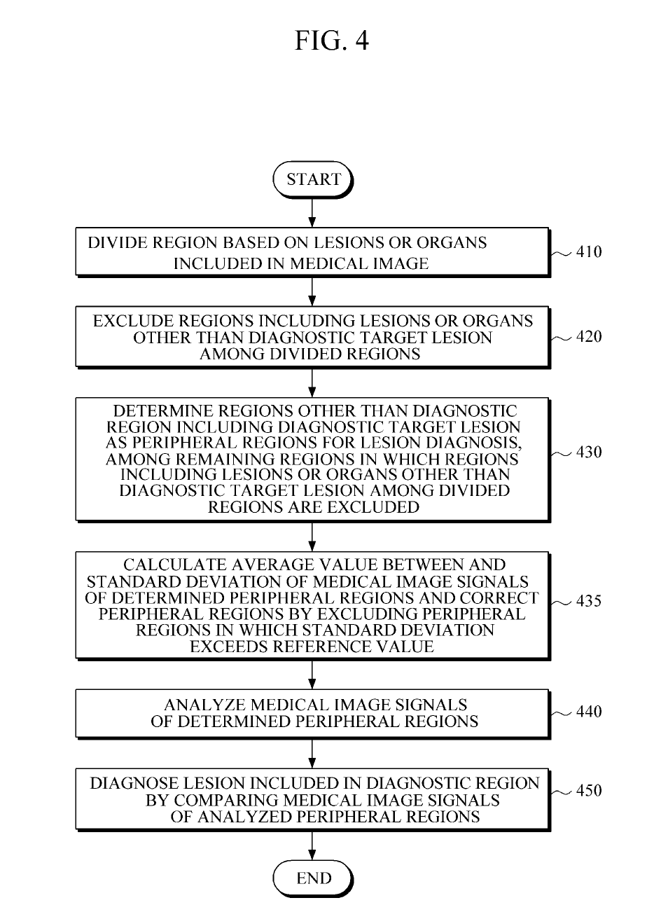

Referring to FIGS. 1 to 4, an example of a lesion diagnosis operation of the example of the lesion diagnosis apparatus using lesion peripheral zone information will be described. FIG. 4 illustrates an example of a lesion diagnosis method using lesion peripheral zone information.

A medical image acquired by an image acquisition apparatus 100 of a CAD system 10 illustrated in FIG. 1 is assumed, and a lesion detection apparatus 200 detects lesions and organs from the medical image is assumed.

In 410, a lesion diagnosis apparatus 300 divides a region based on lesions or organs included in the medical image. Since the region division in which regions are divided into a plurality of regions based on lesions or organs included in the medical image has been described, an explanation of the region division is omitted for conciseness.

In 420, the lesion diagnosis apparatus 300 may exclude regions including lesions or organs other than a diagnostic target lesion among the divided regions in 410. Since the region exclusion in which regions including lesions or organs other than a diagnostic target lesion are excluded has been described, an explanation of the region exclusion is omitted for conciseness.

In 430, the lesion diagnosis apparatus 300 may determine regions other than the diagnostic region including the diagnostic target lesion as peripheral regions for lesion diagnosis, among remaining regions including lesions or organs other than the diagnostic target lesion among the divided regions are excluded. Since the peripheral region determination has already been described, an explanation of the peripheral region determination is omitted for conciseness.

In 440, in response to the peripheral regions being determined in 430, the lesion diagnosis apparatus 300 may analyze medical image signals of the determined peripheral regions. Since the peripheral region determination has already been described, an explanation of the peripheral region determination is omitted for conciseness.

In 450, in response to medical image signals of the peripheral regions being analyzed in 440, the lesion diagnosis apparatus 300 may diagnose a lesion included in the diagnostic region by comparing the medical image signals of the analyzed peripheral regions.

For example, in 450, the lesion diagnosis apparatus 300 may compare medical image signals of peripheral regions located on the rear of the diagnostic region and peripheral regions located on the left and right of the rear of the diagnostic region to diagnose a lesion included in the diagnostic region.

In this case, the lesion diagnosis apparatus 300 may compare an average value of medical image signals of peripheral regions located on the rear of the diagnostic region and an average value of medical image signals of peripheral regions located on the left and right of the rear of the diagnostic region to diagnose a lesion included in the diagnostic region.

A difference between change amounts of medical image signals of peripheral regions located on the rear of the diagnostic region and on the left and right of the rear of the diagnostic region may occur due to a diagnostic target lesion included in the diagnostic region.

Accordingly, the lesion diagnosis apparatus 300 may compare change amounts of medical image signals of peripheral regions located on the rear of the diagnostic region and on the left and right of the rear of the diagnostic region to determine whether a lesion included in the diagnostic region is a cystic mass or a solid mass.

For example, the lesion diagnosis apparatus 300 may compare differences in intensity between medical images of peripheral regions located on the rear of the diagnostic region and on located on the left and right of the rear of the diagnostic region to determine whether a lesion included in the diagnostic region is a cystic mass or a solid mass.

In this case, in response to intensity of medical images of peripheral regions located on the rear of the diagnostic region being greater than intensity of the medical images of the peripheral regions located on the left and right of the rear of the diagnostic region, the lesion diagnosis apparatus 300 may determine a lesion included in the diagnostic region as a cystic mass.

Meanwhile, in response to intensity of medical images of peripheral regions located on the rear of the diagnostic region being less than intensity of the medical images of the peripheral regions located on the left and right of the rear of the diagnostic region, the lesion diagnosis apparatus 300 may determine the lesion included in the diagnostic region as a solid mass.

For example, the lesion diagnosis apparatus 300 may compare differences in brightness between medical images of peripheral regions located on the rear of the diagnostic region and on the left and right of the rear of the diagnostic region to determine whether a lesion included in the diagnostic region is a cystic mass or a solid mass.

In this case, in response to brightness of medical images of peripheral regions located on the rear of the diagnostic region being higher than that of the medical images of the peripheral regions located on the left and right of the rear of the diagnostic region, the lesion diagnosis apparatus 300 may determine a lesion included in the diagnostic region as a cystic mass.

Meanwhile, in response to brightness of medical images of peripheral regions located on the rear of the diagnostic region being lower than that of the medical images of peripheral regions located on the left and right of the rear of the diagnostic region, the lesion diagnosis apparatus 300 may determine a lesion included in the diagnostic region as a solid mass.

As additional aspect, the lesion diagnosis method using lesion peripheral zone information may further includes an operation 435 in which peripheral regions in which the standard deviation exceeds a reference value, between the operations 430 and 440, may be excluded to correct peripheral regions to calculate an average value and a standard deviation of medical image signals of the determined peripheral regions, in 430.

By correcting the peripheral regions, an accuracy of lesion diagnosis may be increased. In other words, in response to a great difference between medical image signals from a particular peripheral region and other peripheral regions due to noise signals existing, excluding regions having the difference may increase the accuracy of lesion diagnosis.

As described above, in response to lesions in a medical image being diagnosed using a PASF method, an accuracy of the lesion diagnosis may be increased where overlapping lesions are present or even sound interference is present due to peripheral lesions.

In addition, based on a consideration of posterior acoustic effects of lesions on the bottom thereof as well as the periphery thereof, the lesions may be diagnosed. Furthermore, in response to organs being on a rear of a lesion or a periphery of the rear of the lesion, a lesion may be diagnosed without influence of the organs.

Accordingly, a diagnostic target lesion may be diagnosed in a state in which lesions or organs around a diagnostic target lesion affecting lesion diagnosis are excluded. Accordingly, the lesion may be more accurately diagnosed and diagnostic reliability may be improved.

The units described herein may be implemented using hardware components and software components. For example, microphones, amplifiers, band-pass filters, audio to digital convertors, and processing devices. A processing device may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such a parallel processors.

The software may include a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. In particular, the software and data may be stored by one or more computer readable recording mediums. The computer readable recording medium may include any data storage device that can store data which can be thereafter read by a computer system or processing device. Examples of the computer readable recording medium include read-only memory (ROM), random-access memory (RAM), CD-ROMs, magnetic tapes, floppy disks, optical data storage devices. Also, functional programs, codes, and code segments for accomplishing the example embodiments disclosed herein can be easily construed by programmers skilled in the art to which the embodiments pertain based on and using the flow diagrams and block diagrams of the figures and their corresponding descriptions as provided herein.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A lesion diagnosis apparatus using lesion peripheral zone information, comprising:
a region division unit configured to divide a region based on lesions or organs included in a medical image;
a region exclusion unit configured to exclude regions including lesions or organs other than a diagnostic target lesion among regions divided by the region division unit; and
a peripheral region determination unit configured to determine regions other than a diagnostic region among remaining regions other than regions excluded by the region exclusion unit, as peripheral regions for lesion diagnosis.

2. The lesion diagnosis apparatus according to claim 1, wherein the region division unit divides the region into units of imaginary quadrangles in which the lesions and organs are inscribed.

3. The lesion diagnosis apparatus according to claim 1, wherein the region exclusion unit further excludes rear regions of the regions including lesions or organs other than the diagnostic target lesion.

4. The lesion diagnosis apparatus according to claim 3, wherein the peripheral region determination unit selects regions located on the rear of the diagnosis region and on the left and right of the rear of the diagnosis region, as peripheral regions, among regions further excluding the rear regions of the regions including lesions or organs other than the diagnostic target lesion.

5. The lesion diagnosis apparatus according to claim 1, further comprising:
an analysis unit configured to analyze medical image signals of the peripheral regions determined by the region determination unit; and
a diagnosis unit configured to diagnose a lesion included in the diagnostic region based on a comparison of medical image signals of the peripheral regions analyzed by the analysis unit.

6. The lesion diagnosis apparatus according to claim 5, wherein the analysis unit calculates an average value and a standard deviation of the medical image signals of the peripheral regions and corrects the peripheral regions by excluding peripheral regions in which the standard deviation exceeds a reference value.

7. The lesion diagnosis apparatus according to claim 5, wherein the analysis unit analyzes change amounts of the medical image signals of the peripheral regions located on the rear of the diagnostic region and on the left and right of the rear of the diagnostic region on the basis of a center of the diagnostic region.

8. The lesion diagnosis apparatus according to claim 7, wherein the change amount of the medical image signals is a difference in intensity between the medical images.

9. The lesion diagnosis apparatus according to claim 7, wherein the change amount of the medical image signals is a difference in brightness between the medical images.

10. The lesion diagnosis apparatus according to claim 7, wherein the analysis unit includes:
a rear region analysis unit configured to analyze medical image signals of the peripheral regions located on the rear of the diagnostic region;
a left and right region analysis unit configured to analyze medical image signals of the peripheral regions located on the left and right of the rear of the diagnostic region, and
a peripheral region correction unit configured to calculate an average value and a standard deviation of the medical image signals of the peripheral regions analyzed by the rear region analysis unit or the left and right region analysis unit and correct peripheral regions by excluding peripheral regions in which the standard deviation exceeds a reference value.

11. The lesion diagnosis apparatus according to claim 5, wherein the diagnosis unit diagnoses the lesion included in the diagnostic region based on a comparison of medical image signals of the peripheral regions located on the rear of the diagnostic region and on the left and right of the rear of the diagnostic region.

12. The lesion diagnosis apparatus according to claim 11, wherein the diagnosis unit determines whether the lesion included in the diagnostic region is a cystic mass or a solid mass based on a comparison of change amounts of the medical image signals of the peripheral regions located on the rear of the diagnostic region and on the left and right of the rear of the diagnostic region.

13. The lesion diagnosis apparatus according to claim 12, wherein the diagnosis unit determines whether the lesion included in the diagnostic region is a cystic mass or a solid mass based on a comparison of differences in intensity between the medical image signals of the peripheral regions located on the rear of the diagnostic region and on the left and right of the rear of the diagnostic region.

14. The lesion diagnosis apparatus according to claim 13, wherein the diagnosis unit determines the lesion included in the diagnostic region as a cystic mass in response to intensity of the medical images of the peripheral regions located on the rear of the diagnostic region being greater than intensity of the medical images of the peripheral regions located on the left and right of the rear of the diagnostic region.

15. The lesion diagnosis apparatus according to claim 13, wherein the diagnosis unit determines the lesion included in the diagnostic region as a solid mass in response to intensity of the medical images of the peripheral regions located on the rear of the diagnostic region being less than intensity of the medical images of the peripheral regions located on the left and right of the rear of the diagnostic region.

16. The lesion diagnosis apparatus according to claim 12, wherein the diagnosis unit determines whether the lesion included in the diagnostic region is a cystic mass or a solid mass based on a comparison of differences in brightness between the medical image signals of the peripheral regions located on the rear of the diagnostic region and on the left and right of the rear of the diagnostic region.

17. The lesion diagnosis apparatus according to claim 16, wherein the diagnosis unit determines the lesion included in the diagnostic region as a cystic mass in response to the brightness of the medical images of the peripheral regions located on the rear of the diagnostic region being higher than that of the medical images of the peripheral regions located on the left and right of the rear of the diagnostic region.

18. The lesion diagnosis apparatus according to claim 16, wherein
the diagnosis unit determines the lesion included in the diagnostic region as a solid mass in response to the brightness of the medical images of the peripheral regions located on the rear of the diagnostic region being lower than that of the medical images of the peripheral regions located on the left and right of the rear of the diagnostic region.

19. The lesion diagnosis apparatus according to claim 11, wherein
the diagnosis unit diagnoses the lesion included in the diagnostic region based on a comparison of an average value of medical image signals of the peripheral regions located on the rear of the diagnostic region and an average value of medical image signals of the peripheral regions located on the left and right of peripheral regions located on the rear of the diagnostic region.

20. A lesion diagnosis method using lesion peripheral zone information, comprising:
dividing a region based on lesions or organs included in a medical image;
excluding regions including lesions or organs other than a diagnostic target lesion among divided regions; and
determining regions other than a diagnosis region including the diagnostic target lesion among remaining regions after excluding the regions including lesions or organs other than the diagnostic target lesion among the divided regions, as peripheral regions for lesion diagnosis.

21. The lesion diagnosis method according to claim 20, further comprising:
analyzing medical image signals of the determined peripheral regions; and
diagnosing a lesion included in the diagnostic region based on a comparison of medical image signals of the analyzed peripheral regions.

22. The lesion diagnosis method according to claim 20, further comprising:
calculating an average value and a standard deviation of the medical image signals of the determined peripheral regions and correcting peripheral regions by excluding peripheral regions in which the standard deviation exceeds a reference value.

23. The lesion diagnosis apparatus according to claim 2, wherein a lesion or organ of the region is surrounded by sides of a quadrangle.

24. The lesion diagnosis apparatus according to claim 2, wherein sides of a quadrangle surround the diagnostic target lesion based on horizontal and vertical lengths of the diagnostic target lesion.
